(19) United States
(12) Patent Application Publication
(10) Pub. No.: US 2025/0215299 A1
(43) Pub. Date: Jul. 3, 2025

(54) NOVEL CRYOGENIC COMPOSITION

(71) Applicants: Patrick LESAGE, SAINT MELOIR DES ONDES (FR); Alexandre LESAGE, RENNES (FR)

(72) Inventors: Patrick LESAGE, SAINT MELOIR DES ONDES (FR); Alexandre LESAGE, RENNES (FR)

(21) Appl. No.: 18/850,881

(22) PCT Filed: Mar. 21, 2023
(86) PCT No.: PCT/FR2023/050394

§ 371 Date: Sep. 25, 2024

(30) Foreign Application Priority Data
Mar. 25, 2022 (FR) 2202675

(51) Int. Cl. C09K 5/06; A61F 7/02

(52) U.S. Cl. CPC C09K 5/063; A61F 7/02; A61F 2007/0214; A61F 2007/0215

(57) ABSTRACT

The present invention relates to a new cryogenic composition intended for use in heat treatment devices, as well as its preparation method. The invention also relates to a heat treatment device, and more particularly to a medical device used to cool a part of the human or animal body, in particular after trauma, inflammation or a surgical procedure. The invention finds application in the therapeutic and/or medical field, but also in other fields such as for example for cooling or maintaining foodstuffs or beverages at low temperatures.

### TECHNICAL FIELD

The present invention relates to a new cryogenic composition intended for use in heat treatment devices, as well as its preparation method. The invention also relates to a heat treatment device, and more particularly to a medical device used to cool a part of the human or animal body, in particular after trauma, inflammation or a surgical procedure. The invention therefore finds application in the therapeutic and/or medical field, but also in other fields such as for example for cooling or maintaining foodstuffs or beverages at low temperatures.

### PRIOR ART

Cryotherapy or cold treatment appeared in the 1970s, and is today widely used in the medical field to heal injured tissues, promote healing, relieve body pain such as sprains, tendinitis, muscle strains. Its principle consists of exposing a part of the human or animal body to cold to cause it to secrete endorphins, which has the effect of causing analgesia, and therefore a lowering of the pain threshold. Cryotherapy is thus used after post-traumatic or post-surgical procedures, for its hemolytic and anti-edematous action.

Cryotherapy uses different sources of cold:
Nitrogen-based devices from the instantaneous gasification of liquid nitrogen, this technique involving the use of heavy and bulky equipment, and therefore unwieldy.
Water-based heat treatment devices. Water is one of the materials that best restores cold. However, at a temperature below 0° C., water turns into ice and has certain disadvantages due to its solid structure which is difficult to conform to an anatomical structure, and the very low temperature of which causes tissue damage.
Devices based on antifreeze compositions which do not have a phase change during freezing at −25° C. These compositions, present in the form of beads or gel, restore a very unstable cold which does not allow effective use in cryotherapy. The devices using these compositions have a surface temperature during thawing which can be of the order of −5° C. for gels and beads.
Phase change devices such as those described in patent EP 1 011 558. This patent describes compositions comprising water, an absorbent product of acrylic polymer type and a humectant. During freezing, part of the water contained in the network of the absorbent product is released and changes phase to transform into ice crystals. When heating, the water re-enters the absorbent network. These compositions remain flexible and conformable. However, they are not very resistant to repeated freezing/thawing cycles, and tend to become less and less flexible as the cycles progress, which makes them uncomfortable and less effective in use. These compositions also have the disadvantage of requiring a container including a semi-permeable water-tight and air-permeable area, which complicates the device. The latter also has, during thawing, a surface temperature below 0° C. for a long period, which further reduces the duration of use of the device because it is necessary to wait for the temperature to stabilize above 0° C. or insert an insulating layer between the device and the skin to use it.

WO2017/125687 describes cryogenic compositions comprising a hydrophobic compound in liquid form having a freezing point below 0° C., in which superabsorbent polymer granules loaded with water and a humectant are immersed. The liquid phase of these compositions is not an emulsion. These cryogenic compositions have good cold kinetics, but insufficient performance in terms of flexibility (aggregation/degradation of superabsorbent polymer granules) beyond a certain number of freezing/thawing cycles.

In this context, the present invention aims at overcoming the disadvantages of the devices of the prior art by proposing a heat treatment device containing a new cryogenic composition which does not alter during freezing/thawing cycles (absence of aggregation/degradation of the cryogenic composition over time). The device of the invention has the advantage of great durability and can thus be reused several times (more than sixty times without alteration). A few minutes after its thawing, the device of the invention has a surface temperature which is ideally between 7 and 10° C., remains stable for a period of at least 70 minutes, and which can go up to 120 minutes. The heat treatment device also remains perfectly flexible after freezing.

The inventors discovered that it was possible to achieve these performances by using a cryogenic composition comprising a significant amount of superabsorbent polymer. They observed that the cryogenic composition of the invention is more stable (resistant to a greater number of freezing/thawing cycles), and more malleable and more flexible after freezing. Unlike the compositions of the prior art, the cryogenic composition of the invention allows more homogeneous absorption of the superabsorbent polymer granules, and thus improves the stability and flexibility of the heat treatment device of the invention.

Another advantage of the invention is the preparation time of the cryogenic composition which is significantly shortened compared to the methods of the prior art, the hydration time of the superabsorbent polymer granules being considerably reduced (divided by two) in the method of the invention, thus leading to a significant saving of time.

Thus, according to a first aspect, the present invention relates to a cryogenic composition comprising:
(a) superabsorbent polymer granules impregnated with a liquid phase, said liquid phase comprising:
(b) at least one hydrophobic compound selected from neopentylene glycol diheptanoate, isopropyl sebacate, isodecyl neopentanoate, isostearyl isostearate, and mixtures thereof,
(c) at least one humectant,
(d) optionally at least one preservative, and
(e) water,
   said superabsorbent polymer granules (a) representing more than 6% by weight relative to the total weight of the cryogenic composition.

Within the meaning of the invention, superabsorbent polymer” means a polymer which is capable, in its dry state, of spontaneously impregnating/absorbing at least 20 times its own weight of aqueous fluid, in particular water. This polymer has a high capacity for absorption and retention of water and aqueous fluids. After absorption of the aqueous liquid, the polymer granules thus soaked in aqueous fluid remain insoluble in the aqueous fluid, and thus retain their individualized state. Examples of superabsorbent polymers are described in the work “Absorbent polymer technology, Studies in polymer science 8” by L. BRANNON-PAPPAS and R. HARLAND, Elsevier edition, 1990.

Among superabsorbent polymers (a) capable of being used in the context of the present invention, mention may be made of crosslinked sodium or potassium polyacrylates, polyacrylamides, copolymers based on ethylene and maleic anhydride, vinyl alcohol copolymers, crosslinked polyethylene oxide, polymers based on starch, gum and cellulose derivatives, pectins, alginates, agar-agar (or agarose), polyethylene amines, polyvinyl amines, and mixtures thereof. The superabsorbent polymer (a) is preferably a crosslinked acrylic homo-or copolymer, and even more preferably a crosslinked sodium or potassium acrylic homo-or copolymer.

Superabsorbent polymer granules (a) are advantageously present in the cryogenic composition of the invention in an amount ranging from 7 to 25%, preferably from 7 to 20%, more preferably from 8 to 15%, even more preferably from 9 to 15%, and even more preferably from 10 to 15%, relative to the total weight of the cryogenic composition.

Advantageously, the superabsorbent polymer granules (a) of the invention are in the form of spherical beads having a diameter of 1 to 6 mm when they are dehydrated. According to an even more preferred embodiment, the spherical beads of superabsorbent polymer (a) are beads of sodium or potassium polyacrylate, and even more preferably of potassium polyacrylate, preferably having a diameter of 1 to 3 mm when they are dehydrated. Said spherical beads can absorb at least 40 times their mass, this characteristic being defined under normal conditions of temperature (20° C.) and pressure (100000 Pa) and for water. Once hydrated, the spherical beads of superabsorbent polymer (a) swell and form soft beads.

In the cryogenic composition of the invention, superabsorbent polymer granules (a) preferably represent at least 7% by weight, and more preferably at least 8% by weight, relative to the total weight of the cryogenic composition. In a particularly preferred embodiment, the superabsorbent polymer granules (a) represent at least 9% by weight, and even more preferably at least 10% by weight, relative to the total weight of the cryogenic composition.

In the cryogenic composition of the invention, the hydrophobic compound (b) advantageously has a freezing point lower than −7° C., preferably lower than −10° C., more preferably lower than −15° C., and even more preferably lower than −20° C. It advantageously has a high molecular weight, ranging from 200 to 700 g.mol⁻¹. The hydrophobic compound (b) is in liquid form. It is preferably selected from neopentylene glycol diheptanoate, isopropyl sebacate, isodecyl neopentanoate, isostearyl isostearate, and mixtures thereof, and more preferably neopentylene glycol diheptanoate. These hydrophobic compounds (b) are marketed for example by the company STÉEARINERIE DUBOIS under the references DUB DNPG (neopentylene glycol diheptanoate, freezing point: −55° C.), DUB DIS (isopropyl sebacate, freezing point: −20° C.), DUB VCI 10 (isodecyl neopentanoate, freezing point: −35° C.), DUB ISIS (isostearyl isostearate), or by the company INTERCHIMIE.

The hydrophobic compound(s) (b) preferably represent 0.1 to 8%, more preferably 0.1 to 6%, and even more preferably 0.1 to 4%, based on the total weight of the cryogenic composition.

In the cryogenic composition of the invention, the humectant (c) can be selected from glycerol, sorbitol, polyethylene glycol, (di)propylene glycol, polypropylene glycol, 1,5-pentanediol, propylene glycol, butylene glycol, diethylene glycol, paraffin oil and mixtures thereof, preferably from glycerol, (di)propylene glycol, polypropylene glycol and mixtures thereof, and even more preferably from (di)propylene glycol, polypropylene glycol and mixtures thereof. Dipropylene glycol is the most preferred humectant (d).

The humectant (c) preferably represents 1 to 20%, more preferably 5 to 20%, and even more preferably 5 to 15%, based on the total weight of the cryogenic composition.

When present, the preservative agent (d) is advantageously selected from isothiazolinones such as 2-methyl-2H-isothiazol-3-one, 5-chloro-2-methyl-4-isothiazolin-3-one, 2-methyl-4-isothiazolin-3-one, and mixtures thereof. Preferably, the preservative agent (d) is 2-methyl-2H-isothiazol-3-one, sold for example under the name Microcare® MT (manufacturer THOR). The preservative agent (d) is advantageously free of formaldehyde compound.

When present, the preservative agent (d) preferably represents 0.05 to 5%, more preferably 0.05 to 4%, and even more preferably 0.1 to 3%, based on the total weight of the cryogenic composition.

The liquid phase of the cryogenic composition of the invention is advantageously in the form of an oil-in-water emulsion. In this oil-in-water emulsion, the fatty phase preferably consists of the hydrophobic compound(s) (b), and the aqueous phase of the humectant(s) (c), optionally the preservative(s) (d), and water (e).

According to a preferred embodiment, the cryogenic composition of the invention comprises:
(a) from 7 to 25%, preferably from 7 to 20%, and more preferably from 8 to 15%, by weight of superabsorbent polymer granules,
(b) from 0.1 to 8%, preferably from 0.1 to 6%, and more preferably from 0.1 to 4%, by weight of at least one hydrophobic compound selected from neopentylene glycol diheptanoate, isopropyl sebacate, isodecyl neopentanoate, isostearyl isostearate, and mixtures thereof,
(c) from 1 to 20%, preferably from 5 to 20%, and more preferably from 5 to 15%, by weight of at least one humectant,
(d) from 0.05 to 5%, preferably 0.05 to 4%, and more preferably 0.1 to 3%, by weight of at least one preservative,
(e) 60 to 90%, preferably 65 to 85%, and more preferably from 70 to 85%, by weight of water,
   said percentages being expressed as percentages by weight relative to the total weight of the cryogenic composition, and the total weight of the cryogenic composition representing 100%.

Another object relates to a method for preparing a cryogenic composition according to the invention comprising the following steps:
(i) with stirring, preparing an oil-in-water emulsion by adding at least one hydrophobic compound (b) in an aqueous mixture based on at least one humectant (c), optionally at least one preservative (d), and water (e),
(ii) adding superabsorbent polymer granules (a) in the oil-in-water emulsion obtained at the end of step (i),
(iii) impregnating the oil-in-water emulsion obtained at the end of step (ii) with the superabsorbent polymer granules (a), allowing said superabsorbent polymer granules (a) to stand at room temperature, preferably until they reach a size comprised between two and four times their initial size.

Step (i) of preparing the oil-in-water emulsion is carried out with stirring, preferably at a speed varying from 5000 to 15000 rpm, and more preferably from 8000 to 12000 rpm, for a duration preferably ranging from 30 seconds to 5 minutes, and more preferably from 30 seconds to 3 minutes. Agitation can be carried out using a Dynamic SMX 800 Turbo mixer.

Step (iii) of impregnating the oil-in-water emulsion obtained at the end of step (ii) with the superabsorbent polymer granules (a) is preferably carried out for a duration ranging from 1 to 5 hours, and more preferably for a duration ranging from 1 to 2 hours.

A heat treatment device comprising a sealed container, inside which is contained a cryogenic composition according to the invention, is also part of the invention. The container is advantageously made of a flexible material such as polyvinyl chloride (PVC), polychloroprene (neoprene), polytetrafluoroethylene (PTFE) or polyethylene (PE), and preferably polyethylene (PE). According to an advantageous embodiment, the container is a multilayer material comprising at least one layer of polyethylene (PE). The device is preferably a sealed and flexible medical bag, which can be applied to a part of the human or animal body, for example to absorb hematomas, edema, or to calm pain.

The heat treatment device of the invention can be obtained by simply filling a sealed container, preferably a medical bag, with a cryogenic composition according to the invention. The cryogenic composition of the invention is preferably manually stirred before being used to fill the sealed container. In practice, the cryogenic composition is generally stored in drums which can be turned over once or twice before use (manual stirring), to homogenize the cryogenic composition before use.

The invention therefore mainly aims at a medical device selected from a facial mask, a splint for the shoulder, elbow, ankle, knee, hip or wrist, and any support comprising a sealed and flexible medical bag according to the invention.

The cryogenic composition of the invention, or the heat treatment device of the invention, can be used to cool a part of the human or animal body, preferably after trauma, inflammation or a surgical procedure. The temperature can be controlled visually using a thermochromic pigment, previously added to the cryogenic composition of the invention. It can also be contained in the material constituting the container. This pigment can be a thermochromic pigment such as those marketed by the company OliKrom.

The heat treatment device of the invention can also be intended for non-medical applications, and be used to lower or maintain the temperature of foodstuffs or beverages such as wine, or to promote their conservation, or for transporting heat-sensitive items in a cold atmosphere. In this case, the temperature can be controlled visually using a thermochromic pigment as described previously.

The heat treatment device of the invention can be used in a heat treatment method, to lower the temperature of a part of the human or animal body or to lower the temperature of foodstuffs or beverages, said method comprising the following steps:
(i′) freezing a device according to the invention at a temperature below 0° C., and preferably at a temperature comprised between −20° C. and −30° C., for a period which may range from 2 to 4 hours,
(ii′) manually kneading the device obtained at the end of step (i′),
(iii′) applying the device obtained at the end of step (ii′) on a part of the human or animal body, using or not a medical device according to the invention, preferably for a duration comprised between 30 minutes and 120 minutes.

In addition to the preceding provisions, the invention also comprises other provisions which will emerge from the additional description which follows, which relates to examples highlighting the advantageous properties of the cryogenic composition of the invention.

### EXAMPLES

### Example 1

A cryogenic composition according to the invention was prepared by mixing 11400 g of water with 1620 g of dipropylene glycol (supplier: INTERCHIMIE) and 30 g of 2-methyl-2H-isothiazol-3-one (Microcare® MT, THOR), under manual stirring. 450 g of neopentylene glycol diheptanoate (DUB DPNG from STÉEARINERIE DUBOIS) were added to the mixture of water, dipropylene glycol and 2-methyl-2H-isothiazol-3-one, then the mixture was stirred at a speed of 11000 rpm for 1 minute and 30 seconds using a Dynamic SMX 800 Turbo mixer to form an oil-in-water emulsion. 1500 g of potassium polyacrylate beads (that is to say 10% by weight relative to the total weight of the composition) having a diameter of 1.5 mm (supplier: Shanghai Chuangshi Medical Technology Group Co., Ltd.) were then added to the emulsion thus obtained. The emulsion was left to stand at room temperature for 1.5 hours (step of impregnating the emulsion by the polymer beads), until the potassium polyacrylate beads had reached an average diameter of 4 mm. 390 g of the mixture thus obtained was poured into a polyethylene (PE) bag of 295 mm in diameter. The bag was then sealed and placed in the freezer at −20° C. for 3 hours. During freezing, part of the water contained in the polymer beads changed phase and transformed into small ice crystals having the appearance of snow.

At the exit from the freezer, the temperature of the sample was measured every minute for 120 minutes under a compression of 14 g.cm⁻², using a TESTO 175 T2 temperature recorder, with a CTN type probe. The measurements were taken every minute at the interface between the frozen bag and a 2 cm thick bag of water at 20° C. The bag temperature remained between 7 and 10° C. for 71 minutes. The bag can therefore be used for cryotherapy treatment for 71 minutes.

The aging of the cryogenic composition was also evaluated by measuring the number of freezing/thawing cycles. Aging becomes visible when the polymer beads begin to aggregate, leading to a decrease in the flexibility of the frozen bag. With the cryogenic composition of Example 1, the bag always remained homogeneous (no degradation of the polymer beads) and flexible until the 60^{th} freezing/thawing cycle. The measurements were stopped at this stage.

### Counter-Example 1

A cryogenic composition representative of the prior art WO2017/125687A1 was prepared by mixing 14000 g of water with 1190 g of dipropylene glycol (supplier: INTERCHIMIE), with manual stirring. 350 g of potassium polyacrylate beads (that is to say 2.2% by weight relative to the total weight of the composition) having a diameter of 1.5 mm (supplier: Shanghai Chuangshi Medical Technology Group Co., Ltd.) were added to the water and dipropylene glycol mixture. The mixture was left to stand at room temperature for 3 hours (step of impregnation of the polymer beads), until the potassium polyacrylate beads reached an average diameter of 5.5 mm. 56 g of neopentylene glycol diheptanoate (DUB DPNG from STÉARINERIE DUBOIS) were poured into a polyethylene (PE) bag 295 mm in diameter. The previously prepared mixture of water, dipropylene glycol and potassium polyacrylate beads was also added to the PE bag. The bag was then sealed and placed in the freezer at −20° C. for 3 hours. During freezing, some of the water contained in the polymer beads changed phase and transformed into small ice crystals with the appearance of snow.

As for example 1, upon exiting the freezer, the temperature of the sample was measured every minute for 120 minutes under a compression of 14 g.cm⁻², using a TESTO 175 T2 temperature recorder, with a CTN type probe. The measurements were taken every minute at the interface between the frozen bag and a 2 cm thick bag of water at 20° C. The temperature of the bag was maintained between 7.5 and 10° C. for 70 minutes.

As for Example 1, the aging of the cryogenic composition of counter-example 1 was evaluated by measuring the number of freezing/thawing cycles. Aging becomes visible when the polymer beads begin to aggregate, leading to a decrease in the flexibility of the frozen bag. The bag deteriorated from the 3^{rd} freezing/thawing cycle (aggregation of the polymer beads), then lost its homogeneity and flexibility and was no longer usable from the 25^{th} freezing/thawing cycle.

### Counter-Example 2

A cryogenic composition was prepared by mixing 12150 g of water with 1620 g of dipropylene glycol (supplier: INTERCHIMIE) and 30 g of 2-methyl-2H-isothiazol-3-one (Microcare® MT, THOR), with manual stirring. 450 g of neopentylene glycol diheptanoate (DUB DPNG from STÉARINERIE DUBOIS) were added to the mixture of water, dipropylene glycol and 2-methyl-2H-isothiazol-3-one, then the mixture was stirred at a speed of 11000 rpm for 1 minute and 30 seconds using a Dynamic SMX 800 mixer to form an oil-in-water emulsion. 750 g of potassium polyacrylate beads (that is to say 5% by weight relative to the total weight of the composition) having a diameter of 1.5 mm (supplier: Shanghai Chuangshi Medical Technology Group Co., Ltd.) were then added to the emulsion thus obtained. The emulsion was left to stand at room temperature for 2.25 hours (polymer bead hydration step), until the potassium polyacrylate beads reached an average diameter of 4.7 mm. 390 g of the mixture thus obtained was poured into a polyethylene (PE) bag of 295 mm in diameter. The bag was then sealed and placed in the freezer at −20° C. for 3 hours. During freezing, part of the water contained in the polymer beads changed phase and transformed into small ice crystals having the appearance of snow.

As for example 1, upon exiting the freezer, the temperature of the sample was measured every minute for 120 minutes under a compression of 14 g.cm⁻², using a TESTO 175 T2 temperature recorder, with a CTN type probe. The measurements were taken every minute at the interface between the frozen bag and a 2 cm thick bag of water at 20° C. The temperature of the bag was maintained between 7 and 10° C. for 70 minutes. The bag can therefore be used for cryotherapy treatment for 70 minutes.

As for example 1, the aging of the cryogenic composition of counterexample 2 was evaluated by measuring the number of freezing/thawing cycles. Aging becomes visible when the polymer beads begin to aggregate, leading to a decrease in the flexibility of the frozen bag. The bag deteriorated from the 7^{th} freezing/thawing cycle (aggregation of the polymer beads), then lost its homogeneity and flexibility and was no longer usable from the 31^{st} freezing/thawing cycle.

The examples above show that the cryogenic composition of Example 1 (invention) significantly improves the flexibility of the frozen bag while retaining the integrity of the polymer beads (absence of aggregation/degradation of the polymer beads) at the same time beyond 60 freeze/thaw cycles. Furthermore, the appearance of the cryogenic composition is improved, since no degradation of the polymer beads is observed. Another advantage of the cryogenic composition of the invention is the shortened impregnation time of the polymer beads, which is reduced to 1.5 hours instead of 3 hours (time saving during the preparation of the cryogenic composition of the invention). Counterexample 2 shows that when the cryogenic composition comprises less than 6% by weight of superabsorbent polymer granules (a) relative to the total weight of the composition, the aging and degradation of the cryogenic composition are accelerated compared to a cryogenic composition according to the invention, with rapid degradation of the polymer beads.

1. A cryogenic composition comprising:
(a) superabsorbent polymer granules impregnated with a liquid phase, said liquid phase comprising:
(b) at least one hydrophobic compound selected from neopentylene glycol diheptanoate, isopropyl sebacate, isodecyl neopentanoate, isostearyl isostearate, and mixtures thereof, and preferably neopentylene glycol diheptanoate,
(c) at least one humectant,
(d) optionally at least one preservative, and
(e) water,
characterized in that said superabsorbent polymer granules (a) represent more than 6% by weight relative to the total weight of the cryogenic composition.

2. The cryogenic composition according to claim 1, characterized in that the superabsorbent polymer granules (a) represent at least 7% by weight, preferably at least 8% by weight, preferably at least 9% by weight, and more preferably at least 10% by weight, relative to the total weight of the cryogenic composition.

3. The cryogenic composition according to claim 1, characterized in that the superabsorbent polymer granules (a) represent an amount ranging from 7 to 25% by weight, preferably from 7 to 20% by weight, more preferably from 8 to 15% by weight, even more preferably 9 to 15% by weight, and even more preferably 10 to 15% by weight, relative to the total weight of the cryogenic composition.

4. The cryogenic composition according to claim 1, characterized in that it comprises:
(a) from 7 to 25%, preferably from 7 to 20%, more preferably from 8 to 15%, even more preferably from 9 at 15%, and even more preferably from 10 to 15%, by weight of superabsorbent polymer granules,
(b) from 0.1 to 8%, preferably from 0.1 to 6%, and more preferably from 0.1 to 4%, by weight of at least one hydrophobic compound selected from neopentylene glycol diheptanoate, isopropyl sebacate, isodecyl neopentanoate, isostearyl isostearate, and mixtures thereof, and preferably neopentylene glycol diheptanoate,
(c) from 1 to 20%, preferably from 5 to 20%, and more preferably from 5 to 15%, by weight of at least one humectant,
(d) from 0.05 to 5%, preferably from 0.05 to 4%, and more preferably 0.1 to 3%, by weight of at least one preservative agent,
(e) 60 to 90%, preferably 65 to 85%, and more preferably 70 at 85%, by weight of water,
said percentages being expressed as percentages by weight relative to the total weight of the cryogenic composition, and the total weight of the cryogenic composition representing 100%.

5. The cryogenic composition according to claim 1, characterized in that the superabsorbent polymer of the granules (a) is selected from crosslinked sodium or potassium polyacrylates, polyacrylamides, copolymers based on ethylene and maleic anhydride, vinyl alcohol copolymers, crosslinked polyethylene oxide, polymers based on starch, gum and cellulose derivatives, pectins, alginates, agar-agar (or agarose), polyethylene amines, polyvinyl amines, and mixtures thereof.

6. The cryogenic composition according to claim 1, characterized in that said superabsorbent polymer granules (a) are in the form of spherical beads having a diameter of 1 to 6 mm when they are dehydrated, said spherical beads being able to absorb at least 40 times their mass.

7. The cryogenic composition according to claim 6, characterized in that said superabsorbent polymer granules (a) are beads of sodium or potassium polyacrylate, or a mixture thereof, advantageously of potassium polyacrylate, having a diameter of 1 to 3 mm when they are dehydrated.

8. The cryogenic composition according to claim 1, characterized in that the humectant agent (c) is selected from glycerol, sorbitol, polyethylene glycol, (di)propylene glycol, polypropylene glycol, 1,5-pentanediol, propylene glycol, butylene glycol, diethylene glycol, paraffin oil, and mixtures thereof, preferably from glycerol, (di)propylene glycol, polypropylene glycol and mixtures thereof, more preferably from (di)propylene glycol, polypropylene glycol and mixtures thereof, and even more preferably dipropylene glycol.

9. The cryogenic composition according to claim 1, characterized in that the preservative agent (d) is selected from isothiazolinones such as 2-methyl-2H-isothiazol-3-one, 5-chloro-2-methyl-4-isothiazolin-3-one, 2-methyl-4-isothiazolin-3-one, and mixtures thereof, and preferably 2-methyl-2H-isothiazol-3-one.

10. A method for preparing a cryogenic composition as defined according to claim 1, characterized in that it comprises the following steps:
(i) with stirring, preparing an oil-in-water emulsion by adding at least one hydrophobic compound (b) in an aqueous mixture based on at least one humectant (c), optionally at least one preservative (d), and water (e),
(ii) adding superabsorbent polymer granules (a) in the oil-in-water emulsion obtained at the end of step (i),
(iii) impregnating the oil-in-water emulsion obtained at the end of step (ii) with the superabsorbent polymer granules (a), allowing said superabsorbent polymer granules (a) to stand at room temperature, preferably until they reach a size comprised between two and four times their initial size.

11. The method according to claim 10, characterized in that step (iii) of impregnating the oil-in-water emulsion obtained at the end of step (ii) with the superabsorbent polymer granules (a) is carried out for a duration ranging from 1 to 5 hours, and preferably for a duration ranging from 1 to 2 hours.

12. A heat treatment device, characterized in that it comprises a sealed container, inside which is contained a cryogenic composition as defined according to claim 1.

13. The device according to claim 12, characterized in that said container is a sealed and flexible medical bag made of a material selected from polyvinyl chloride (PVC), polychloroprene (neoprene), polytetrafluoroethylene (PTFE) or polyethylene (PE), and preferably made of a multilayer material comprising at least one layer of polyethylene (PE).

14. A medical device selected from a facial mask, a splint for the shoulder, elbow, ankle, knee, hip or wrist, and any support comprising a sealed and flexible medical bag as defined according to claim 13.

15. A use of a cryogenic composition as defined according to claim 1, to lower or maintain the temperature of foodstuffs or beverages, or to promote their conservation, or for transporting heat-sensitive items in a cold atmosphere.
